(19) United States
(12) Patent Application Publication
(10) Pub. No.: US 2023/0351591 A1
(43) Pub. Date: Nov. 2, 2023

(54) METHOD FOR DETECTING MUTATIONS AND RELATED NON-TRANSITORY COMPUTER STORAGE MEDIUM

(71) Applicant: TAIPEI MEDICAL UNIVERSITY, TAIPEI CITY (TW)

(72) Inventors: CHENG-YU CHEN, TAIPEI CITY (TW); QUOC KHANH LE NGUYEN, TAIPEI CITY (TW)

(21) Appl. No.: 18/299,041

(22) Filed: Apr. 11, 2023

(30) Foreign Application Priority Data
Apr. 27, 2022 (TW) 111116053

(51) Int. Cl. G06T 7/00; G06T 7/11; G06V 10/25; G06V 10/44; G16H 50/20

(52) U.S. Cl. CPC G06T 7/0012; G06T 7/11; G06V 10/25; G06V 10/44; G16H 50/20; G06T 2207/10081; G06T 2207/20064; G06T 2207/20132; G06T 2207/30061; G06T 2207/30096

(57) ABSTRACT

The present disclosure relates to a method for detecting a mutation and a related non-transitory computer storage medium. Some embodiments of the present disclosure relate to a method for detecting a mutation. The method includes: receiving a computed tomography (CT) image of a lung; generating a first set of radiomics features based on the CT image through a first image processing model; determining a first region of the CT image through a segmentation model; generating a second set of radiomics features based on the first region of the CT image; and determining whether a mutation occurs based on the first and second sets of radiomics features through a classifier model.

### CROSS-REFERENCE TO RELATED APPLICATIONS

This application claims a benefit and priority to TW Invention Patent Application Serial No. 111116053, filed on Apr. 27, 2022, which is hereby incorporated by reference in its entirety.

### BACKGROUND OF THE INVENTION

### 1. Field of the Invention

The present disclosure generally relates to a method for detecting a mutation and a related non-transitory computer storage medium, and in particular, to a method for detecting a mutation based on a computed tomography (CT) image of a lung and a related non-transitory computer storage medium.

### 2. Description of the Related Art

Lung cancer is a malignant tumor that is the most lethal and second most common, which causes about 1.8 million deaths, nearly one fifth of all deaths resulted from cancer in 2020. Non-small cell lung cancer (NSCLC) is the main type. A five-year survival rate of NSCLC patients with tumors in the lung is about 63%. A five-year survival rate of patients with cancer cell metastasis as low as 7%. Surgery is infeasible for the patients with cancer cell metastasis. Therefore, use of gene drive and multimodality therapies is vital for molecular analysis of a tumor tissue and other related analysis.

### SUMMARY OF THE INVENTION

Further classification of molecular subtypes, which contributes to development of treatment and prognosis of the lung cancer, is considered as a direction for further research. Specifically, molecular analysis of mutation statuses of driver genes (such as an epidermal growth factor receptor (EGFR) gene and a Kirsten rat sarcoma virus (KRAS) gene) and exon levels (such as T790M, L858R, and an exon-19 deletion) becomes a method for treating the lung cancer. The molecular analysis is of biological and clinical significance for the lung cancer.

Artificial intelligence (AI) has great potential in many health fields, such as data analysis and drug discovery in biomedicine. “Useful” data may be separated from massive aggregated data through AI. Health data may be explored through a modern supercomputer and a machine learning system, so as to pre-determine a disease condition for more effective treatment. The new science of pharmacogenomics offers a possibility of precision drugs. An early human disease, especially the early lung cancer, can be diagnosed and predicted through fully trained algorithms by virtue of AI. An AI system can quickly learn to refine key information and make a decision based on the information. Therefore, an AI-based lung cancer detection platform needs to be developed for diagnosis of the early lung cancer. Moreover, an AI-based mutation detection platform needs to be developed for diagnosis of the early lung cancer.

Radiomics means fusion of a medical image with genetic characteristics of a human tumor. The radiomics can realize non-invasive diagnosis and prognosis. A core concept of the radiomics is to provide effective therapeutic, prognostic, or predictive information through a model that includes biological or medical data. The radiomics model attracts many researchers in related fields to engage in researches. For example, many radiomics researches have been launched for a topic of predicting a mutation in the EGFR, the KRAS, an anaplastic lymphoma kinase (ALK), or a BRAF (a human gene used for encoding a B-Raf protein).

Although significant achievements have been made in prediction and classification in recent years, improvements are still required. In particular, classification of molecular subtypes of the lung cancer is still a challenging topic, and a large amount of data is needed to support the classifier model. In addition, previous researches ignored advantages of deep learning in the radiomics in improving the prediction effect. The prediction effect can be effectively improved by using the new method provided in the present disclosure.

The present disclosure may include but is not limited to:
automatically segmenting an image region including a lung nodule (or a lung tumor) through big data training and an advanced AI model;
generating radiomics features according to a lung segmentation model;
improving prediction effect of CT radiogenomics for lung cancer patients through deep learning, feature selection, and the radiomics; and
classifying EGFR mutation statuses in the lung cancer patients at an exon level.

Some embodiments of the present disclosure relate to a method for detecting a mutation. The method includes: receiving a computed tomography (CT) image of a lung; generating a first set of radiomics features based on the CT image through a first image processing model; determining a first region of the CT image through a segmentation model; generating a second set of radiomics features based on the first region of the CT image; and determining whether a mutation occurs based on the first and second sets of radiomics features through a classifier model.

Some embodiments of the present disclosure relate to a method for detecting the mutation described above. The method further includes: determining whether an epidermal growth factor receptor (EGFR) mutates.

Some embodiments of the present disclosure relate to a method for detecting the mutation described above. The method further include: determining whether a T79M mutation occurs; determining whether an L858R mutation occurs; and determining whether an exon-19 deletion occurs.

Some embodiments of the present disclosure relate to a non-transitory computer storage medium, storing a plurality of program instructions. The program instructions, when executed by a processor, cause a plurality of operations to be performed. The plurality of operations include: receiving a computed tomography (CT) image of a lung; generating a first set of radiomics features based on the CT image through a first image processing model; determining a first region of the CT image through a segmentation model; generating a second set of radiomics features based on the first region of the CT image; and determining whether a mutation occurs based on the first and second sets of radiomics features through a classifier model.

All models, techniques, and statistical analysis in the present disclosure may be implemented by using a Python programming language, a scikit-learn library, and a Tensorflow framework.

### BRIEF DESCRIPTION OF THE DRAWINGS

FIG. 1 illustrates a system according to some embodiments of the present disclosure.

FIG. 2 illustrates a flowchart according to some embodiments of the present disclosure.

FIG. 3 illustrates a recursive feature elimination result according to some embodiments of the present disclosure.

FIG. 4A shows a flowchart according to some embodiments of the present disclosure.

FIG. 4B shows a flowchart according to some embodiments of the present disclosure.

FIG. 5 illustrates a flowchart according to some embodiments of the present disclosure.

In order to better understand the above implementations disclosed herein and additional implementations and embodiments thereof, reference should be made to the following implementations with reference to the above drawings. In the drawings, the similar reference symbols indicate similar elements.

### PREFERRED EMBODIMENT OF THE PRESENT INVENTION

A method, a system, and other implementations of the present invention are described. Reference is made to some embodiments of the present invention, examples of which are illustrated in the drawings. Although the present invention is described combination with the embodiments, it should be understood that the present invention is not limited to the specific embodiments. Rather, the present invention is intended to cover alternatives, modifications, and equivalents within the spirit and scope of the present invention. Therefore, the description and the drawings should be viewed in an illustrative sense rather than in a restrictive sense.

In addition, numerous specific details are stated in the following description to provide a thorough understanding of the present invention. However, a person of ordinary skill in the art can practice the present invention without the specific details. In other cases, methods, processes, operations, components, and networks known to a person of ordinary skill in the art are not described in detail to avoid confusion as to the implementations of the present invention.

Some embodiments of the present disclosure are described in detail below with reference to the drawings.

In recent years, a new research direction of cancers has emerged, which focuses on a relationship between imaging phenotypes and genomics. The research direction is referred to as “radiogenomics” (which is a combination of radiomics and genomics). The radiogenomics generally means a relationship between imaging features (such as a photographic phenotype or a radiophenotype) of a disease and gene expression patterns, gene mutations, and other genome-related features. Therefore, radiomics features may be extracted from a computed tomography (CT) image and a diagnosis may be made based on the features. The radiogenomics may be used for resolve problems about different cancers such as a brain cancer, a breast cancer, and a lung cancer.

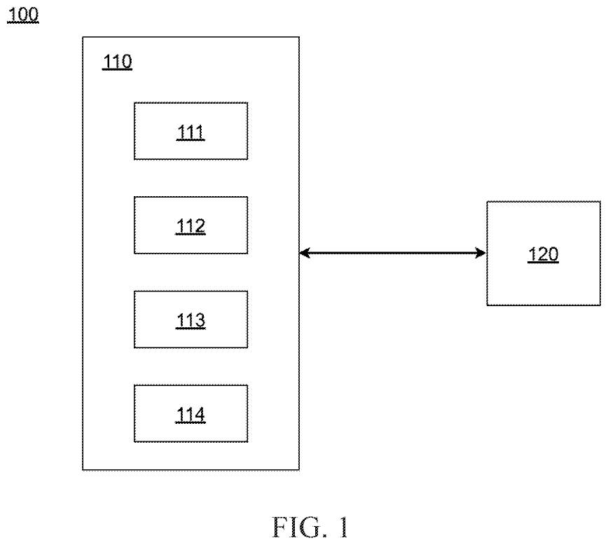

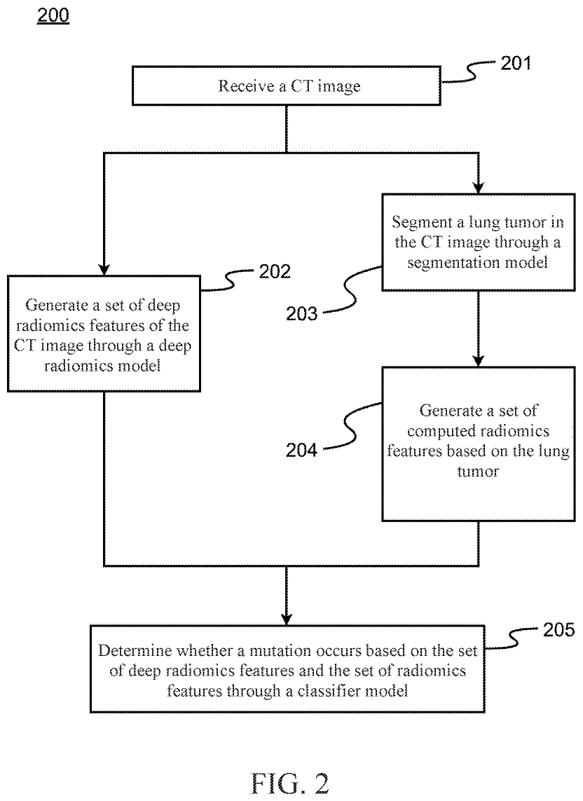

FIG. 1 illustrates an exemplary embodiment of a computer system 100 that can perform one or more operations of the method of the present disclosure. In at least some embodiments of the present disclosure, the computer system 100 may include a computing device 110 and a database 120. The computing device 110 may be a server computer, a client computer, a personal computer (PC), a tablet PC, a set-top box (STB), a personal digital assistant (PDA), a mobile phone, a smart phone, or any other suitable computing devices. The computing device 110 includes a processor 111, an input/output interface 112, a communication interface 113, and a memory 114. The database 120 may be configured to store a medical image (such as the CT image) and related information. The database 120 may store to-be-analyzed medical images. The input/output interface 112 is coupled to the processor 111. A user may cause the computing device 110 to perform the operations or a method described in the present disclosure (such as a method in FIG. 2 to FIG. 5) after permission by the input/output interface 112. The communication interface 113 may be coupled to the processor 111. The computing device 110 may communicate with the database 120 through the communication interface 113. The communication interface 113 and the database 120 is compatible with one or more of the following communication protocols: a universal serial bus (USB), an Ethernet, Bluetooth, IEEE 802.11, 3GPP long term evolution (LTE) (4G), and 3GPP new radio (NR) (5G). The memory 114 may be a non-transitory computer-readable storage medium. The memory 114 may be coupled to the processor 111. The memory 114 may store program instructions executable by one or more processors (for example, the processor 111). The program instructions stored on the memory 114, when executed, may cause one or more operations of the method disclosed in the present disclosure to be performed. As another exemplary embodiment, the program instructions may cause the computing device 110 to perform a method of detecting a mutation described in the present disclosure.

The model disclosed in the present disclosure collects data from hospitals in Taiwan, China, and retrieves radiogenomics (which is a combination of radiomics and genomics) data about the lung cancer. A total of 1,000 CT images of lung cancer patients are retrieved to train the model. The retrospectively collected data set may be from lung cancer patients who had a surgical diagnosis and an EGFR mutation/exon gene detection. In addition, lung cancer patients are retrieved from published data to validate the significance of the model disclosed in the present disclosure, and the model is evaluated based on data about different populations.

FIG. 2 illustrates a flowchart according to some embodiments of the present disclosure. FIG. 2 illustrates a flowchart of a method 200. The method 200 may include an operation 201, an operation 202, an operation 203, an operation 204, and an operation 205. The method 200 includes two subprocesses. One of the subprocesses includes the operation 202, and the other of the subprocesses includes the operation 203 and the operation 204. The computing device 110 may perform the method 200.

In the operation 201, a CT image is received. The computing device 110 may receive the CT image. The computing device 110 may receive the CT image from the database 120.

In the operation 202, a set of deep radiomics features of the CT image may be generated through a deep radiomics model.

The “deep radiomics features” are introduced in the method disclosed in the present disclosure. The deep radiomics features are generated by using a deep transfer learning method. A trained deep learning model captures significant features (such as the radiomics features) from the original CT image. By virtue of high efficiency of the deep learning model in extracting information from images, the deep radiomics features disclosed in the present disclosure can further improve the effect of the method for predicting a mutation.

The EfficientNet has pretty high efficiency in different computer vision tasks. In some embodiments of the present disclosure, the EfficientNet is used as the deep radiomics model to generate a set of deep radiomics features of the CT image.

During the training, the CT image is directly inputted into the EfficientNet model to understand how the model learns through the data. Then a set of deep radiomics features may be obtained from the EfficientNet model. The set of deep radiomics features are then combined with the corresponding set of computed radiomics features described below for analysis of gene mutation (such as an EGFR mutation).

In the operation 203, a lung tumor in the CT image may be segmented through a segmentation model. In some embodiments, a plurality of regions of interest (ROI) in the CT image may be obtained through segmentation by the segmentation model, for example, one or more regions including the lung tumor. In the operation 202, the ROI segmentation may be performed on the original CT image to locate the lung tumor.

Completing the automatic segmentation through the segmentation model in the operation 203 may have advantages such as reducing a time spent by a radiologist in reading the image and providing accurate segmentation. The segmentation model may be implemented through different techniques such as machine learning (that is, a conventional algorithm) and deep learning (that is, a convolutional neural network, such as the U-NET).

The present disclosure discloses a novel segmentation model. A workflow of the segmentation model may further include data pre-processing. During the data pre-processing, the CT image may be cropped into 64×64×64 cubes to reduce complexity of an input into the model. A Hounsfield unit (HU) of the CT image may be standardized to be between −2000 and 2000.

In some embodiments, U-NET models of different forms may be separately trained to obtain prediction probabilities. The prediction probabilities are inserted into another fully connected layer to be mixed to obtain the segmentation model. For example, a threshold for determining a boundary of a predicted segmented pixel may be set to 0.6. The performance of the segmentation model obtained by integrating the U-NET models of different forms in the present disclosure is superior to the performance of a single model. The regions including the lung tumor (that is, the ROIs) are segmented from the CT image by using the segmentation model to generate a set of computed radiomics features in the operation 204.

The radiogenomics may be used for resolve problems about different cancers. In the present disclosure, a plurality of features may be further extracted based on the regions generated by the segmentation model in the operation 204. In some embodiments of the present disclosure, in the operation 204, radiomics features of nine different forms including original, wavelet-HHH, wavelet-HHL, wavelet-HLH, wavelet-HLL, wavelet-LHH, wavelet-LHL, wavelet-LLH, and wavelet-LLL may be used. L indicates a low frequency signal, such as a low frequency signal of the image, and H indicates a high frequency signal, such as a high frequency signal of the image. In some embodiments, after the ROIs are segmented from the CT image by the segmentation model, in the operation 204, a set of computed radiomics features may be determined based on at least one of the ROIs of the CT image, an HHH wavelet transform region of the ROIs, an HHL wavelet transform region of the ROIs, an HLH wavelet transform region of the ROIs, an HLL wavelet transform region of the ROIs, an LHH wavelet transform region of the ROIs, an LHL wavelet transform region of the ROIs, an LLH wavelet transform region of the ROIs, and an LLL wavelet transform region of the ROIs.

In some embodiments, each of the nine different forms of radiomics features used in the above operation 204 may include six subclasses of features. The six subclasses of features may include a first-order feature, a gray level co-occurrence matrix (GLCM) feature, a gray level size zone matrix (GLSZM) feature, a gray level run length matrix (GLRLM) feature, a neighboring gray tone difference matrix (NGTDM) feature, and a gray level dependence matrix (GLDM) feature. In some embodiments, the set of computed radiomics features generated in the operation 204 may further include other subclasses disclosed in a PyRadiomics package of a python programming language.

In the operation 205, whether a gene mutation occurs may be determined through a classifier model based on the set of deep radiomics features generated in the operation 202 and the set of computed radiomics features generated in the operation 204.

In some embodiments, the set of deep radiomics features and the set of computed radiomics features may be combined into a unique set, from which a machine learning classifier model may generate a prediction result (such as a lung cancer class or a mutation status class). In some embodiments, a feature set may be formed by selecting better features from the set of deep radiomics features and the set of computed radiomics features. For example, a feature set may be formed by selecting features with better prediction results from the set of deep radiomics features and the set of computed radiomics features.

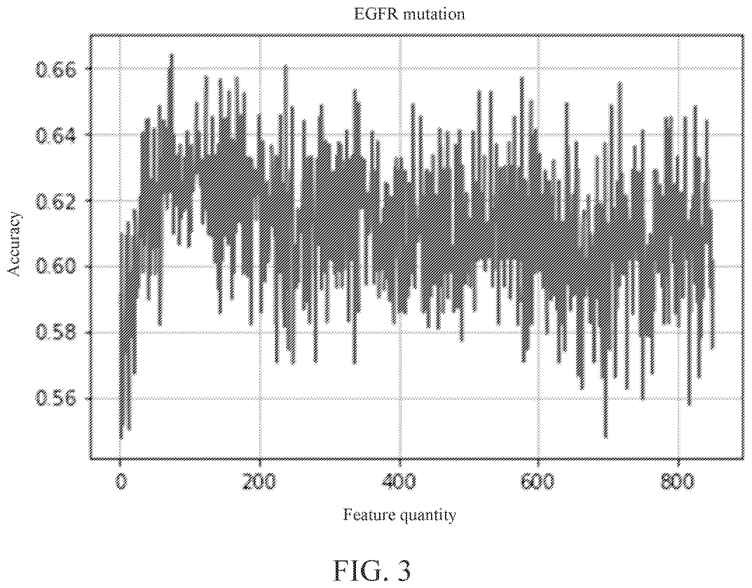

In some embodiments, recursive feature elimination (RFE) may be performed to find an optimal feature set. More specifically, the plurality of features may be sorted and inputted into the classifier model one by one to obtain a cut-off point. Features from which the cut-off point can be obtained are considered as an optimal feature. FIG. 3 discloses an RFE result for obtaining the optimal feature set according to some embodiments of the present disclosure. As shown in FIG. 3, the classifier model of the present disclosure can achieve an optimal performance with about 80 features.

Another novel characteristic of the present disclosure is the generation of the prediction result based on the deep learning model. Therefore, the present disclosure can be used for interpreting hidden information of the image and thus assist in diagnosis of the lung cancer when applied to the radiomics. In addition to generating the set of computed radiomics features from the lung tumor (through the segmented ROIs) as mentioned above, hidden features (such as the set of deep radiomics features) in the CT image may be further extracted through the deep learning algorithm (such as the EfficientNet). The set of deep radiomics features outputted by the deep learning model in the present disclosure are different from the set of computed radiomics features generated from the lung tumor. The combination of the deep learning, the radiomics, the genomics, and clinical features can effectively improve the effect of the prediction model. Therefore, the system, the device, or the method of the present disclosure is a diagnosis and prediction framework for the lung cancer based on the deep radiogenomics.

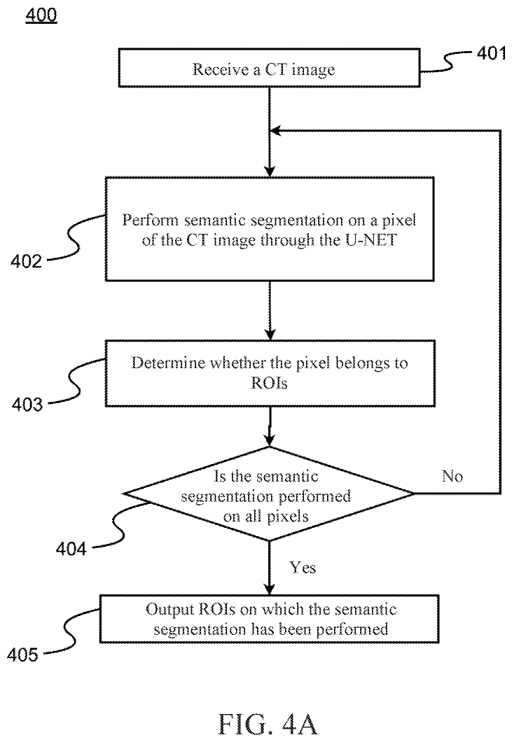

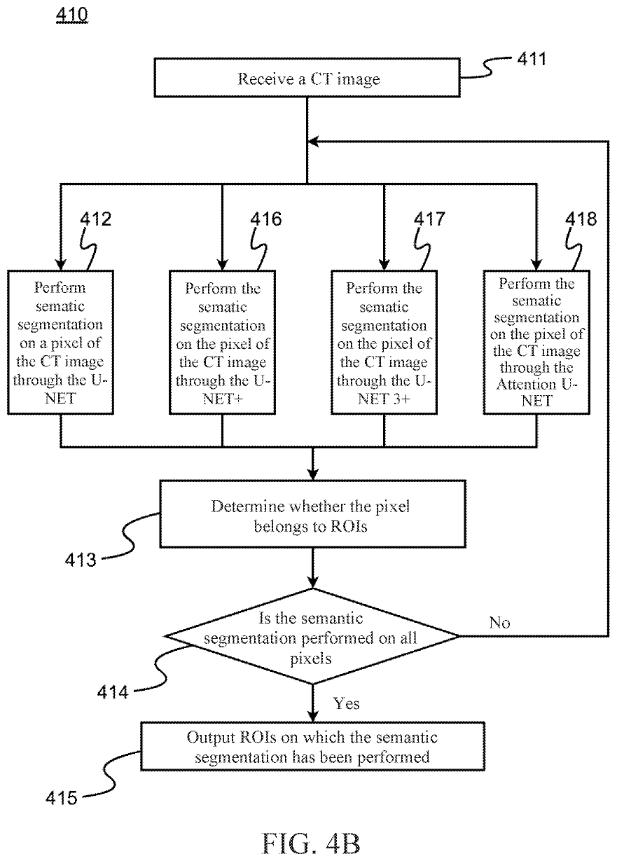

FIG. 4A shows a flowchart of a semantic segmentation method 400 according to some embodiments of the present disclosure. The method 400 may be performed in the operation 202 and the method 400 may be implemented by the segmentation model. The method 400 may be performed by the computing device 110. The method 400 may include an operation 401, an operation 402, an operation 403, an operation 404, and an operation 405.

In the operation 401, a CT image is received. The computing device 110 may receive the CT image. The computing device 110 may receive the CT image from the database 120.

In the operation 402, semantic segmentation may be performed on a pixel of the CT image through the U-NET model. The model used in the operation 402 may be replaced with other models adapted to perform the semantic segmentation of the CT image.

In the operation 403, whether the pixel belongs to the ROIs may be determined based on a result of the operation 402. The ROIs may be regions including a lung tumor or a lung nodule.

In the operation 404, whether the semantic segmentation is performed on all pixels of the CT image may be determined. In the operation 404, it may be determined whether determining as to belonging to the ROIs or not is performed for all of the pixels of the CT image. If the semantic segmentation is not performed on all of the pixels of the CT image, the operation 402 and the operation 403 may be performed on a next pixel of the CT image. If the semantic segmentation is performed on all of the pixels of the CT image, the operation 405 may be performed on the next pixel of the CT image.

In the operation 405, ROIs in the CT image on which the semantic segmentation has been performed may be outputted. In the operation 204, a set of computed radiomics features may be further extracted from the ROIs on which the semantic segmentation has been performed.

FIG. 4B shows a flowchart of a semantic segmentation method 410 according to some embodiments of the present disclosure. The method 410 may be performed in the operation 202 and the method 410 may be implemented by the segmentation model. The method 410 may be performed by the computing device 110. The method 410 may include an operation 411, an operation 412, an operation 413, an operation 414, an operation 415, an operation 416, an operation 417, and an operation 418.

In the operation 411, a CT image is received. The computing device 110 may receive the CT image. The computing device 110 may receive the CT image from the database 120.

In the operation 412, semantic segmentation may be performed on a pixel of the CT image by using the U-NET model. In the operation 416, the semantic segmentation may be performed on the pixel of the CT image through the U-NET++ model. In the operation 417, the semantic segmentation may be performed on the pixel of the CT image through the U-NET 3+ model (or referred to as the U-NET +++ model or the U-NET 3 Plus model). In the operation 418, the semantic segmentation may be performed on the pixel of the CT image through the Attention U-NET model. The models used in the operation 412, the operation 416, the operation 417, and the operation 418 may be replaced with other models adapted to perform the semantic segmentation on the CT image.

In operation the 413, whether the pixel belongs to the ROIs may be determined based on results of the operation 412, the operation 416, the operation 417, and the operation 418. The ROIs may be regions including a lung tumor or a lung nodule.

In some embodiments, the operation 413 may further include the following operations. After the models used in the operation 412, operation 416, the operation 417, and the operation 418 are trained, accuracies of the four models may be obtained, that is: a %, b %, c %, and d %. If a given pixel is determined to be a pixel of the ROIs in one of the operation 412, the operation 416, the operation 417, and the operation 418, “1” may be outputted for the given pixel in the operation. If a given pixel is determined not to be a pixel of the ROIs in one of the operation 412, the operation 416, the operation 417, and the operation 418, “0” may be outputted for the given pixel in the operation. For example, in the operation 412, the operation 416, the operation 417, and the operation 418, “1”, “1”, “0”, and “1” may be respectively outputted for a given pixel. The four outputs may be further inputted into the fully connected layer. For example, the fully connected layer may output: 1×a %+1×b %+0×c %+1×a %. When the output of the fully connected layer is greater than or equal to a threshold, the given pixel may be determined to be a pixel of the ROIs. In some embodiments, the threshold may be set to be in a range of 0.5 to 0.8. In some embodiments, the threshold may be set to 0.6.

In the operation 414, whether the semantic segmentation is performed on all pixels of the CT image may be determined. In the operation 414, it may be determined whether determining as to belonging to the ROIs or not is performed for all of the pixels of the CT image. If the semantic segmentation is not performed on all of the pixels of the CT image, the operation 412, the operation 416, the operation 417, the operation 418, and the operation 413 may be performed on a next pixel of the CT image. If the semantic segmentation is performed on all of the pixels of the CT image, the operation 415 may be performed on the next pixel of the CT image.

In the operation 415, ROIs in the CT image on which the semantic segmentation has been performed may be outputted. In the operation 204, a set of computed radiomics features may be further extracted from the ROIs on which the semantic segmentation has been performed.

The machine learning technology may be used be used in the research of the radiomics. By virtue of complex interactions between the plurality of features and between feature combinations and clinical endpoints being researched, the machine learning technology can handle high-dimensional radiomics feature sets with higher robustness and can build effective prognosis/prediction models compared with conventional statistical analysis. Therefore, supervised machine learning models including a random forest (RF), extreme gradient boosting (XGBoost), and a support vector machine (SVM) may be used for binary classification. In some embodiments of the present disclosure, based on the performance, the XGBoost may be a relatively desirable classifier model. For the classifier model of the present disclosure, different feature selection technologies may be used, to reduce a quantity of features and avoid complexity and overfitting of the model. For example, RFE may be used to find an optimal feature set.

In some embodiments of the present disclosure, 5-fold cross-validation may be used for validating the method for predicting a mutation and the related models disclosed in the present disclosure. The 5-fold cross-validation disclosed in the present disclosure may ensure that each observation from the original data set can appear in training and testing sets. Therefore, the 5-fold cross-validation disclosed in the present disclosure has a smaller deviation than other verification methods. For the binary classification, the performance of the method for predicting a mutation and the related models disclosed herein is evaluated by using relevant values such as an area under curve (AUC), a sensitivity (SN), a specificity (SP), and an accuracy (ACC) of a receiver operating characteristic (ROC). The relevant values indicate percentages of correct predictions for different data sets (such as positive, negative, and entire data).

Predicting a status of an EGFR mutation, especially T790M, L858R, and an exon-19 deletion, is of great significance for the diagnosis and treatment of the early lung cancer. For example, the EGFR mutation is the most common molecular subtype of the lung cancer. This is because a large amount of genomic data is required to implement the prediction method or the prediction model for all EGFR exon levels. Prior to the present disclosure, the radiomics research has not implemented the prediction method or the prediction model for all of the EGFR exon levels. Specifically, T790M, L858R, and an exon-19 deletion are considered as essential biomarkers for the diagnosis and treatment of the lung cancer. The present disclosure may fill the above knowledge gap. In addition, the classifier model of the present disclosure may be a multi-label classifier model that can accurately predict the EGFR mutation status of the selected exon level.

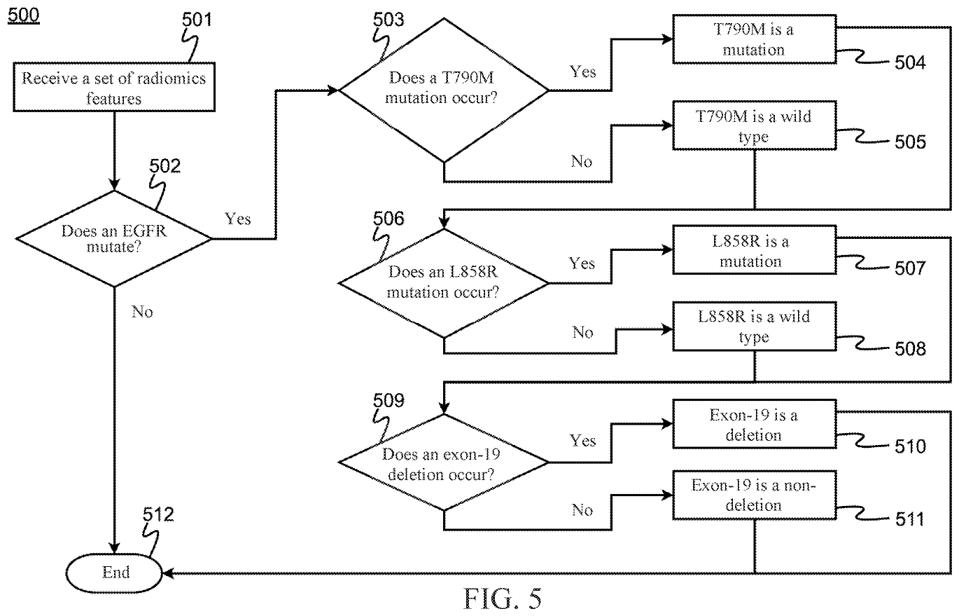

FIG. 5 illustrates a flowchart of a classification method 500 according to some embodiments of the present disclosure. The method 500 may be performed in the operation 205, and the method 500 may be implemented by the classifier model. The method 500 may be performed by the computing device 110. The method 500 may include an operation 501, an operation 502, an operation 503, an operation 504, an operation 505, an operation 506, an operation 507, an operation 508, an operation 509, an operation 510, an operation 511, and an operation 512.

In the operation 501, a set of radiomics features are received. The computing device 110 may be configured to receive the set of radiomics features. The device 110 may receive the set of radiomics features from the database 120. The set of radiomics features received in the operation 501 may be a unique combination of a set of deep radiomics features and a set of computed radiomics features. The set of radiomics features received in the operation 501 may alternatively be a set of deep radiomics features or a set of computed radiomics features.

In the operation 502, whether an EGFR mutates may be determined. If the EGFR mutates, the operation 503 is performed. If the EGFR does not mutate, the operation 512 is performed and the method 500 is ended. In some embodiments, a result of the determination in the operation 502 may be recorded through a flag. For example, if the EGFR mutates, the corresponding flag is set to “1”, and if the EGFR does not mutate, the corresponding flag is set to “0”.

In the operation 503, whether a T790M mutation occurs may be determined, if the T790M mutation occurs, the operation 504 is performed, and if T790M mutation does not occur, the operation 505 is performed.

In the operation 504, if the T790M mutation occurs, “T790M is a mutation” may be recorded. For example, in the operation 504, the recording may be realized through a flag, and if T790M is a mutation, the corresponding flag is set to “1”. After the operation 504, the operation 506 may be performed.

In the operation 505, if the T790M mutation does not occur, “T790M is a wild type” may be recorded. For example, in the operation 505, the recording may be realized through a flag, and if T790M is the wild type, the corresponding flag is set to “0”. After the operation 505, the operation 506 may be performed.

In the operation 506, whether an L858R mutation occurs may be determined, if the L858R mutation occurs, the operation 507 is performed, and if the L858R mutation does not occur, the operation 508 is performed.

In the operation 507, if the L858R mutation occurs, “L858R is a mutation” may be recorded. For example, in the operation 507, the recording may be realized through a flag, and if the L858R is a mutation, the corresponding flag is set to “1”. After the operation 507, the operation 509 may be performed.

In the operation 508, if no L858R mutation occurs, “L858R is a wild type” may be recorded. For example, in the operation 508, the recording may be realized through a flag, and if L858R is the wide type, the corresponding flag is set to “0”. After the operation 508, the operation 509 may be performed.

In the operation 509, whether an exon-19 deletion occurs may be determined, if the exon-19 deletion occurs, the operation 510 is performed, and if the exon-19 deletion does not occur, the operation 511 is performed.

In the operation 510, if the exon-19 deletion occurs, “exon-19 is a deletion” may be recorded. For example, in the operation 510, the recording may be realized through a flag, and if exon-19 is a deletion, the corresponding flag is set to “1”. After the operation 510, the operation 512 may be performed, and method 500 is ended.

In the operation 511, if no exon-19 deletion occurs, “exon-19 is a non-deletion” may be recorded. For example, in the operation 511, the recording may be realized through a flag, and if exon-19 is a non-deletion, the corresponding flag is set to “0”. After the operation 511, the operation 512 may be performed, and method 500 is ended.

The present disclosure discloses a significant method and system for detecting gene mutations. The method and system for detecting gene mutations of the present disclosure are significant for the lung cancer. According to the present disclosure, the development of the deep learning and auto-radiogenomics models are used in the radiomics. Current researches demonstrated the biological and clinical significance of molecular typing in the lung cancer. Therefore, the radiogenomics offers many opportunities for non-invasive diagnosis and prognostic prediction of the lung cancer. The present disclosure creates an accurate CT radiogenomics model for the EGFR mutation. The present disclosure can provide more helpful information for drug selection and prediction and drug response prediction for the lung cancer patients.

Although the present invention has been described and illustrated with reference to the specific embodiments of the present disclosure, the description and illustration do not limit the present invention. A person skilled in the art should understand that various changes and substitutive equivalents may be made without departing from the true spirit and scope of the present invention as defined by the scope of the appended claims. The description may not be drawn to scale. An artistic representation in this application may differ from an artistic representation in an actual invention due to manufacturing processes and tolerances. Other embodiments of the present invention that are not specifically described may exist. The description and drawings should be considered as illustrative rather than restrictive. Modifications may be made so that a particular situation, material, composition of matter, method, or process adapts to the objective, spirit, and scope of the present invention. All modifications fall within the scope of the appended claims. While the methods disclosed herein have been described with reference to particular operations performed in a particular order, it should be understood that the operations may be combined, subdivided, or reordered without departing from the teachings of the present invention to form equivalent methods. Therefore, unless otherwise particularly indicated herein, the order and grouping of operations are not limited in the present invention. Furthermore, the effects detailed in the above embodiments and their analogs are merely examples. Therefore, this application may further have other effects.

In addition, the logical processes illustrated in the drawings do not necessarily require the particular order or sequential order that is shown to achieve the desired result. In addition, additional steps may be provided, or steps may be eliminated from the illustrated process, and additional components may be added to or removed from the illustrated system. Therefore, other embodiments all fall within the scope of the attached claims.

What is claimed is:

1. A method for detecting a mutation, comprising:
receiving a computed tomography (CT) image of a lung;
generating a first set of radiomics features based on the CT image through a first image processing model;
determining a first region of the CT image through a segmentation model;
generating a second set of radiomics features based on the first region of the CT image; and
determining whether a mutation occurs based on the first and second sets of radiomics features through a classifier model.

2. The method according to claim 1, wherein
the CT image is cropped into a plurality of cubes, each of the cubes comprising 64×64×64 pixels; and
a hounsfield unit (HU) of the CT image is standardized to be between −2000 and 2000.

3. The method according to claim 1, wherein the first image processing model comprises the EfficientNet.

4. The method according to claim 1, wherein the segmentation model comprises the U-Net.

5. The method according to claim 4, wherein
the segmentation model further comprises the U-Net+, the U-Net 3+, and the Attention U-Net; and
the first region of the CT image is determined based on outputs of the U-Net, the U-Net+, the U-Net3, and the Attention U-Net.

6. The method according to claim 1, wherein
the second set of radiomics features are determined based on the first region of the CT image, an HHH wavelet transform region of the first region, an HHL wavelet transform region of the first region, an HLH wavelet transform region of the first region, an HLL wavelet transform region of the first region, an LHH wavelet transform region of the first region, an LHL wavelet transform region of the first region, an LLH wavelet transform region of the first region, and an LLL wavelet transform region of the first region.

7. The method according to claim 1, wherein
the second set of radiomics features comprises a plurality of first-order features, a plurality of gray level co-occurrence matrix (GLCM) features, a plurality of gray level size zone matrix (GLSZM) features, a plurality of gray level run length matrix (GLRLM) features, a plurality of neighboring gray tone difference matrix (NGTDM) features, and a plurality of gray level dependence matrix (GLDM) features.

8. The method according to claim 1, wherein
the classifier model comprises a random forest (RF), extreme gradient boosting (XGBoost), and a support vector machine (SVM).

9. The method according to claim 1, further comprising:
determining whether an epidermal growth factor receptor (EGFR) mutates.

10. The method according to claim 9, further comprising:
determining whether a T79M mutation occurs;
determining whether an L858R mutation occurs; and
determining whether an exon-19 deletion occurs.

11. A non-transitory computer storage medium, storing a plurality of program instructions, the program instructions, when executed by a processor, causing a set of operations to be performed, the operations comprising:
processing a computed tomography (CT) image of a lung through a first image processing model, to determine a first set of radiomics features of the CT image;
processing the CT image through a segmentation model, to determine a first region of the CT image;
processing the CT image to calculate a second set of radiomics features of the first region of the CT image; and
determining whether a mutation occurs based on the first and second sets of radiomics features through a classifier model.

12. The non-transitory computer storage medium according to claim 11, wherein
the CT image is cropped into a plurality of cubes, each of the cubes comprising 64×64×64 pixels; and
a hounsfield unit (HU) of the CT image is standardized to be between −2000 and 2000.

13. The non-transitory computer storage medium according to claim 11, wherein the first image processing model comprises the EfficientNet.

14. The non-transitory computer storage medium according to claim 11, wherein the segmentation model comprises the U-Net.

15. The non-transitory computer storage medium according to claim 14, wherein
the segmentation model further comprises the U-Net+, the U-Net 3+, and the Attention U-Net; and
the first region of the CT image is determined based on outputs of the U-Net, the U-Net+, the U-Net3, and the Attention U-Net.

16. The non-transitory computer storage medium according to claim 11, wherein
the second set of radiomics features are determined based on the first region of the CT image, an HHH wavelet transform region of the first region, an HHL wavelet transform region of the first region, an HLH wavelet transform region of the first region, an HLL wavelet transform region of the first region, an LHH wavelet transform region of the first region, an LHL wavelet transform region of the first region, an LLH wavelet transform region of the first region, and an LLL wavelet transform region of the first region.

17. The non-transitory computer storage medium according to claim 11, wherein
the second set of radiomics features comprises a plurality of first-order features, a plurality of gray level co-occurrence matrix (GLCM) features, a plurality of gray level size zone matrix (GLSZM) features, a plurality of gray level run length matrix (GLRLM) features, a plurality of neighboring gray tone difference matrix (NGTDM) features, and a plurality of gray level dependence matrix (GLDM) features.

18. The non-transitory computer storage medium according to claim 11, wherein
the classifier model comprises a random forest (RF), extreme gradient boosting (XGBoost), and a support vector machine (SVM).

19. The non-transitory computer storage medium according to claim 11, further comprising
determining whether an epidermal growth factor receptor (EGFR) mutates.

20. The non-transitory computer storage medium according to claim 19, further comprising:
determining whether a T79M mutation occurs;
determining whether an L858R mutation occurs; and
determining whether an exon-19 deletion occurs.
